(19) United States
(12) Patent Application Publication
(10) Pub. No.: US 2014/0079675 A1
(43) Pub. Date: Mar. 20, 2014

(54) Repair of Neurodegenerative Diseases

(71) Applicant: HumaCell, Inc., Rochester, NY (US)

(72) Inventor: Kurt Osther, Scottsdale, AZ (US)

(73) Assignee: HumaCell, Inc., Rochester, NY (US)

(21) Appl. No.: 14/110,029

(22) PCT Filed: Mar. 30, 2012
(86) PCT No.: PCT/EP12/55816

§ 371 Date: Nov. 21, 2013

Related U.S. Application Data
(60) Provisional application No. 61/516,703, filed on Apr. 4, 2011.

(51) Int. Cl. A61K 35/48; A61K 48/00; A61K 35/14; A61K 35/50; A61K 35/28; A61K 35/12

(52) U.S. Cl. CPC A61K 35/51; A61K 35/28; A61K 35/35; A61K 35/14; A61K 35/50; A61K 48/00
USPC 424/93.21; 424/93.7; 514/44 R

(57) ABSTRACT

Disclosed are methods and means for treatment of neurodegenerative disease. In particular, the methods entail administration of stem cells expressing genes encoding therapeutically effective amounts of Nrf2. Also disclosed is a gene-therapy method for treatment of neurodegenerative disease, where genes encoding Nrf2 are administered.

### FIELD OF THE INVENTION

The present invention relates to the field of therapy of neurodegenerative diseases. In particular, the present invention relates to methods and means for re-establishing expression of genes encoding Nrf2 in order to treat or ameliorate neurodegenerative diseases.

### BACKGROUND OF THE INVENTION

### Amyotrophic Lateral Sclerosis (ALS)

ALS is the most common adult-onset motor neuron disease, caused by the progressive degeneration of motor neurons in the spinal cord, brainstem, and motor cortex (Rowland LP et al., N Engl J Med 2005, 344: 1688-1700).

Approximately 10-20% of familial ALS is caused by a toxic gain-of-function induced by mutations of the Cu/Zn-superoxide dismutase (SOD1). Rodents over-expressing mutated forms of hSOD1 generally develop an ALS-like phenotype. Although the molecular mechanism underlying this toxic gain-of-function remains unknown, toxicity to motor neurons requires mutant SOD1 expression in non-neuronal cells as well as in motor neurons (Rosen DR et al., Nature 1993, 362: 59-62; Gurney M E et al., Science 1994, 264: 1772-1775; Howland D S et al., Proc Natl Acad Sci USA 2002, 99: 1604-1609; Clement A M et al., Science 2003, 302: 113-117).

### Astrocytes and Neurodegenerative Disease

Astrocytes are protecting cells such as the neurons in the brain. Among other things, they are capable of mediating several immunological/inflammatory including antioxidation effects (Filosa J A et al., Nat Neurosci 2006, 9: 1397-1403; Takano T et al., Nat Neurosci 2006, 9: 260-267; Kim J H et al., J Biochem Mol Biol 2006, 39:339-345).

Expression of various toll like receptors (TLR) on astrocytes endows the ability to recognize not only bacterial and viral signals but also endogenous “danger” signals such as heat shock proteins, fibrinogen degradation products, and free DNA (Konat G W et a/., J Neurochem 2006, 99: 1-12).

Physiologically, astrocytes play an important protective role against infection, generating inflammatory cytokines such as TNF-alpha, IL-1beta, and IL-6 (Wen L L et al., Exp Neurol 2007, 205: 270-278).

Through secretion of various chemokines such as CXCLIO, CCL2 and BAFF, astrocytes play an important role in shaping adaptive immune responses in the CNS (Farina C et al., Trends Immunol 2007, 28: 138-145).

Astrocytes have antigen presenting capabilities and have been demonstrated to activate T and B cell responses against exogenous and endogenous antigens (Carpentier P A et a/., Glia 2005, 49: 360-374; Constantinescu C S et al., J Neurochem 2005, 95: 331-340).

Although astrocytes play a critical role against CNS infection, these cells also have the potential of causing damage to the host when functioning in an aberrant manner. For example, various neurological diseases are associated with astrocyte overproduction of inflammatory agents, which causes neural malfunction or death. In amyotrophic lateral sclerosis (ALS), astrocyte secretion of a soluble neurotoxic substance has been demonstrated to be involved in disease progression (Holden C, Science 2007, 316: 353; Nagai M et al., Nat Neurosci 2007, 10: 615-622).

Astrocyte hyperactivation has been demonstrated in this disease by imaging, as well as autopsy studies (Johansson A et al., J Neurol Sci 2007, 255: 17-22; Yokota O et al., Acta Neuropathol (Berl) 2006, 112: 633-645; Schiffer D et al., J Neurol Sci 1996, 139(Suppl): 27-33).

In multiple sclerosis, astrocytes play a key role in maintaining autoreactive responses and pathological plaque formation (Bologa L et al., Brain Res 1985, 346: 199-203; Petzold A et al., Mult Scler 2004, 10: 281-283).

In stroke, activated astrocytes contribute to opening of the blood brain barrier, as well as to secretion of various neurotoxic substances that contribute to post infarct neural damage (Pekny M et al., Neurology 2003, 60: 548-554).

Vargas et a/, compared brain autopsy samples from 11 autistic children with 7 age-matched controls. It was demonstrated that an active neuroinflammatory process takes place in the cerebral cortex, white matter, and notably in cerebellum of autistic patients both by immunohistochemistry and morphology. Importantly, astrocyte production of inflammatory cytokines was observed, including production of cytokines known to affect various neuronal functions such as TNF-alpha and MCP-1. CSF samples from living autistic individuals but not controls also displayed upregulated inflammatory cytokines as demonstrated by ELISA (Vargas D L et a/., Ann Neurol 2005, 57: 67-81).

### Inflammatory Cytokines and Genetic Factors in Neurodegenerative Disease

The potent effects of inflammatory cytokines on neurological function cannot be underestimated. For example, patients receiving systemic IFN-gamma therapy for cancer, even though theoretically the protein should not cross the blood brain barrier, report numerous cognitive and neurological abnormalities (Huang D et al., Immunol Rev 2000, 177: 52-67; Loftis J M et al., J Affect Disord 2004, 82: 175-190).

In fact, IFN-gamma, one of the products of activated astrocytes, has been detected at elevated levels in the plasma of children with autism (Huang D et al., Immunol Rev 2000, 177: 52-67; Stubbs G, J Autism Dev Disord 1995, 25: 71-73; Sweeten T L et al., Biol Psychiatry

Mechanistically, inflammatory mediators mediate alteration of neurological function through a wide variety of different pathways, either directly or indirectly altering neuron activity. For example, the common neurotoxin used in models of Parkinson's Disease, M PTP, is believed to mediate its activity through activation of IFN-gamma production, leading to direct killing of dopaminergic neurons in the substantia nigra. This is evidenced by reduced MPTP neuronal toxicity in IFN-gamma knockout mice or by addition of blocking antibodies to IFN-gamma (Mount M P et al., J Neurosci 2007, 27: 3328-3337).

T cell and B cell abnormalities have been reported systemically in autistic children. These have included systemic T cell lymphopenia, weak proliferative responses to mitogens, and deranged cytokine production (Cohly H H et al, Int Rev Neurobiol 2005, 71: 317-341; Yonk U et al., Immunol Left 1990, 25: 341-345).

In terms of indirect effects of IFN-gamma, it is known that this cytokine activates the enzyme 2,3-indolaminedeoxygenase, leading to generation of small molecule neurotoxins such as the kynurenine metabolites (an L-tryptophan metabolite), 30H-kynurenine and quinolinic acid, which have been implicated in dementias associated with chronic inflammatory states (Sardar A M et al., Neurosci Lett 1995, 187: 9-12; Brown R R et al., Adv Exp Med Biol 1991, 294: 425-435).

Autoimmune-like pathophysiology appears to be prevalent in autism and several lines of reasoning suggest that it may be causative. Firstly, numerous types of autoantibodies have been detected in children with autism but not in healthy or mentally challenged controls.

These include antibodies to myelin basic protein, brain extracts, Purkinje cells and glial cells in extracted peptides, neutrophic factors, and neuron-axon filament and glial fibrillary acidic protein (Singh V K et al., Brain Behav Immun 1993, 7:97-103; Silva S C et al., J Neuroimmunol 2004, 152: 176-182; Singer H S et al., J Neuroimmunol 2006, 178: 149-155; Vojdani A etal., Nutr Neurosci 2004, 7:151-161; Connolly A M etal., Biol Psychiatry 2006, 59:354-363; Kozlovskaia G V etal., Zh Nevrol Psikhiatr Im S S Korsakova 2000, 100:50-52).

Secondly, family members of autistic children have a higher predisposition towards autoimmunity compared to control populations (Sweeten T L et al., Pediatrics 2003, 112:e420; Comi A M etal., J Child Neurol 1999, 14:388-394).

Hinting at genetic mechanisms are observations that specific HLA haplotypes seem to associate with autism (Warren R P eta/., J Neuroimmunol 1996, 67:97-102; Daniels W W et al., Neuropsychobiology 1995, 32:120-123).

Another genetic characteristic associated with autism is a null allele for the complement component C4b (Warren R P etal., Clin Exp Immunol 1991, 83:438-440).

Both HLA haplotypes as well as complement component gene polymorphisms have been strongly associated with autoimmunity (Muller-Hilke B eta/., Curr Pharm Des 2006, 12:3743-3752; Moulds J M etal., Front Biosci 2001, 6:D986-991; Yu C Y etal., Prog Nucleic Acid Res Mol Biol 2003, 75:217-292).

### Oxidative Stress and Neurodegenerative Disease

Oxidative stress is induced by a vast range of factors including xenobiotics, drugs, heavy metals, UV lights and ionizing radiation (Rushmore T H etal., J Biol Chem. 1991, 266:11632-11639). Oxidative stress leads to the generation of Reactive Oxygen Species (ROS) and electrophiles. ROS and electrophiles generated can have a profound impact on survival, growth development and evolution of all living organisms (Breimer L H, Mol Carcinog. 1990, 3:188-197; Meneghini R, Free Radic Biol Med. 1997, 23:783-792). ROS include both free radicals, such as the superoxide anion and the hydroxyl radical, and oxidants such as hydrogen peroxide (Jaiswal A K., Free Radic Biol Med. 2004;36:1199-1207). ROS and electrophiles can cause diseases such as cancer, cardiovascular complications, acute and chronic inflammation, and neurodegenerative diseases (Breimer L H. supra). Therefore, it is obvious that cells must constantly labor to control levels of ROS, preventing them from accumulation.

Initial effects of oxidative/electrophilic stress leads often to activation of a battery of defensive gene expression that leads to detoxification of chemicals and ROS and prevention of free radical generation and cell survival. Of these genes, some are enzymes such as NAD(P)H: quinine oxidoreductase 1 (NQO1), NRH: quinone oxidoreductase 2 (NQ02), glutathione S-transferase Ya subunit (GST Ya Subunit), heme oxygenase 1(HO-1), and γ-glutamylcysteine synthetase (γ-GCS), also known as glutamate cysteine ligase (GCL). Other genes have end products that regulate a wide variety of cellular activities including signal transduction, proliferation, and immunologic defense reactions.

### Nrf2 and Neurodegenerative Disease

Nrf2 is a nuclear transcription factor that controls the expression and coordinated induction of a battery of defensive genes encoding detoxifying enzymes and antioxidant proteins. This is a mechanism of critical importance for cellular protection and cell survival and identified to be for instance an integrate part of astrocytes and astroglia, which surround various portions of the cerebral endothelium and play a critical role in regulating perfusion as well as regulating the blood brain barrier function. Nrf2 appears to be a master regulator of the endogenous antioxidant response, which is critical in defending cells against environmental insults and maintaining intracellular redox balance. The novel role for Nrf2 in promoting neuronal cell differentiation opens new perspectives for therapeutic uses of Nrf2 or Nrf2 activators in patients with neurodegenerative diseases (Zhao F et al. Free Radic Biol Med 2009, 47: 867-879).

Nrf2 is a part of the family of human genes encoding basic leucine zipper (bZIP) transcription factors and belongs to the Cap N Collar (CNC-bZIP) subfamily with an implication in adaptive responses to oxidative stress (M P Chan et al., Proc Nat Acad Sci. 91: 9926-9930, 1994). Nrf2 is a basic leucine zipper transcription factor that can bind to a NF-E2/AP-1 repeat sequence in the promoter of the beta-globin gene and has two types of binding partners, a cytoplasmic repressor (Keap 1) and multiple nuclear binding partners.

Both Nrf2 and HO-1 levels were increased and co-localized with reactive astrocytes in the degenerating lumbar spinal cord of rats expressing the ALS-linked SOD 1 G93A mutation. Overexpression of Nrf2 in astrocytes increased survival of co-cultured embryonic motor neurons and prevented motor neuron apoptosis mediated by nerve growth factor through p75 neurotrophin receptor. Taken together, these results emphasize the key role of astrocytes in determining motor neuron fate in amyotrophic lateral sclerosis.

Nrf2 and its downstream proteins have shown to have important functions in protection against oxidative stress or chemical-induced cellular damage in liver and lung, prevention in cancer formation in GI tract, promotion of the wound-healing process (S Braun et al., Mol. Cell. Biol. 2002, 22:5492-5505).

According to Johnson et al. disruption of Nrf2 resulted in a more severe clinical course, a more rapid onset, and a greater percentage of mice with the disease. Furthermore, increased immune cell infiltration and glial cell activation in spine was observed. In conjunction, there was observed increased inflammatory enzyme (iNOS, phox-47, gp91-phox, and phox-67), cytokine (IFN-gamma, IL1-b, TNF-alpha, and IL-12), and chemokine (BLC and MIG) gene expression levels in the Nrf2-deficient mice compared to the WT mice, supporting the notion that Nrf2 can modulate an autoimmune neuroinflammatory response.

These results show that the absence of Nrf2 exacerbates the development of EAE suggesting that activation of Nrf2 may then attenuate pathogenesis of autoimmune diseases such as MS as well as other neurodegenerative diseases that present with neuroinflammation (Johnson DA et al. Toxicol Sci. 2010, 114(2): 237-46).

Nrf2 may be retained in the cytoplasm by an inhibitor “INrf2”, which is a cytosolic inhibitor (INrf2), also known as Keap 1 (Kelch-like ECH-associating protein 1) appearing as a dimer, which retains Nrf2 in the cytoplasm of the cell such as for instance in the astrocyte (Zipper L M, M ulcahy RT. The Keap 1 BTB/POZ dimerization function is required to sequester Nrf2 in cytoplasm. J Biol Chem. 2002; 277: 36544-36552). In response to oxidative/electrophilic stress, Nrf2 is switched on and then off by distinct early and delayed mechanisms.

Oxidative/electrophilic modification of cysteine-151 in INrf2cysteine and/or PKC phosphorylation of serine-40 in Nrf2 results in the escape or release of Nrf2 from INrf2. Nrf2 is stabilized and translocates to the nucleus, forms heterodimers with unknown proteins, and binds antioxidant response element (ARE) that leads to coordinated activation of gene expression. It takes less than fifteen minutes from the time of exposure to switch on nuclear import of Nrf2.

It is increasingly probable that overexpression of Nrf2 in astrocytes in some important manner confers the protection on motor neurons, which in ALS patients apparently is impaired to such a degree that Nrf2 together with ARE cannot induce the expression of the downstream factors and thereby not send signals to glutathione secretion and glutamate dehydrogenase secretion to prevent a probable excess of glutamate, which among others can be detrimental to the motor neurons in diseases such as ALS.

In vitro and in vivo studies have shown a role of Nrf2 in neuroprotection and protection against Parkinson's disease.

Johnson et a/, transplanted Nrf2-overexpressing astrocytes into the mouse corpus striatum prior to introducing lesions with malonate. This procedure led to dramatic protection against malonate-induced neurotoxicity (Johnson J A et a/. Ann N Y Acad Sci. 2008, 1147: 61-69). It was found that in cell cultures the results obtained from the astrocyte-motor neuron co-culture indicated how important it was that the astrocytes secrete glutathione as a component that should be secreted by astrocytes as a main component of the neuroprotection conferred by Nrf2 overexpression. If the astrocytes do not produce or release Nrf2 from the cytoplasm to enter the nucleus of the cells and most probably to connect with ARE, followed by the activation of the downstream factors, it appears from the Johnson et a/.'s studies that absent neuroprotection of the other brain cells, these cells may very well be exposed to oxidation stress, etc. and will eventually get impaired or even enter into apoptosis.

Vargas et a/, described in 2008 the findings in transgenic mice over-expressing Nrf2 selectively in astrocytes using the glial fibrillary acidic protein (GFAP) promoter that the toxicity of astrocytes expressing ALS-linked mutant hSOD1 to co-cultured motor neurons was reversed by Nrf2 overexpression, demonstrating that Nrf2 activation in astrocytes significantly delayed the onset of neurodegeneration and delayed the onset of degeneration as well as extending the survival of the challenged mice. Vargas et a/, concluded that Nrf2 activation in astrocytes may be a viable therapeutic target to prevent chronic neurodegenerative diseases. The group focused in this publication from 2008 especially on the crucial importance of the Nrf2 activity present in astrocytes, which protects for instance motor neurons in Amyotrophic Lateral

Overexpression of Nrf2 in astrocytes has also been shown to increase survival of co-cultured embryonic motor neurons and prevent motor neuron apoptosis mediated by nerve growth factor through p75 neurotrophin receptor. Taken together, these results emphasize the key role of astrocytes in determining motor neuron fate in amyotrophic lateral sclerosis.

It has recently been shown that transcriptional activation of protective genes is mediated by a cis-acting element called the antioxidant responsive element (“ARE”). The transcription factor Nrf2 (NF-E2-related factor 2) binds to the ARE. Thus, activation of the Nrf2/ARE pathway protects cells from oxidative stress-induced cell death in the brain. The pathogenesis of various chronic neurodegenerative diseases including Alzheimer's, Parkinson's, and Huntington's diseases and amyotrophic lateral sclerosis (ALS) is associated with increased oxidative stress with neuronal cell death during the pathogenesis of these diseases.

Hence it has been hypothesized that activation of Nrf2 and/or the Nrf2/ARE pathway appears to be a novel neuroprotective pathway that confers resistance to a variety of oxidative stress-related neurodegenerative insults. In recent studies performed by the Johnson study group from the Division of Pharmaceutical Sciences, University of Wisconsin, Madison, Wis., primary neuronal cultures treated with chemical activators of the Nrf2-ARE pathway displayed significantly greater resistance to oxidative stress induced neurotoxicity.

In disseminated multiple sclerosis (MS), the autoimmune part of this disease may also in an indirect manner be augmented by Nrf2 modulation. MS is considered an autoimmune disease characterized by peripheral activation of CD4(+) T cells that migrate into the central nervous system (CNS) and mount an autoimmune neuroinflammatory attack on myelin and oligodendrocytes. However, as described by Johnson et a/., the generation of inflammatory-mediated reactive oxygen and nitrogen species secreted by persistently activated microglia, and secondary to these events, is equally destructive in MS. Nrf2-modulation in an acute autoimmune model of multiple sclerosis, called EAE was produced in wild type mice and Nrf2-knock out (Nrf2-KO) mice, which were immunized with myelin oligodendrocyte glycoprotein (MOG35-55. It was found that in the Nrf2-knock-out mice a more rapid onset of EAE was noted, and a greater percentage of the mice came down with this acute autoimmune multiple sclerosis model, which led to the conclusion that the absence of Nrf2 exacerbates the development of EAE and thus suggested that activation of Nrf2 may attenuate pathogenesis of autoimmune diseases such as multiple sclerosis as well as other neurodegenerative diseases (Johnson D A et al., Toxicol Sci. 2009, 114: 237-246).

### Glutamate Dehydrogenase

Glutamate is the major excitatory amino acid of the mammalian brain but can be toxic to neurons if its extracellular levels are not tightly controlled. Astrocytes have a key role in the protection of neurons from glutamate toxicity, through regulation of extracellular glutamate levels via glutamate transporters and metabolic and antioxidant support. It has been reported that that cultures of rat astrocytes incubated with high extracellular glutamate (5 mM) exhibit a twofold increase in the extracellular concentration of the tripeptide antioxidant glutathione (GSH) over 4 h. Incubation with glutamate did not result in an increased release of lactate dehydrogenase, indicating that the rise in GSH was not because of membrane damage and leakage of intracellular pools. Glutamate-induced increase in extracellular GSH was also independent of de novo GSH synthesis, activation of NMDA and non-NMDA glutamate receptors or inhibition of extracellular GSH breakdown.

Dose-response curves indicate that GSH release from rat astrocytes is significantly stimulated even at 0.1 mM glutamate. The ability of astrocytes to increase GSH release in the presence of extracellular glutamate could be an important neuroprotective mechanism enabling neurones to maintain levels of the key antioxidant, GSH, under conditions of glutamate toxicity (Frade J et al., J Neurochem. 2008 May, 105 (4): 1144-52).

The exact mechanisms that regulate glutamate fluxes through this pathway, however, have not been fully understood. In the human, glutamate dehydrogenase exists in heat-resistant and heat-labile isoforms, encoded by the GLUD 1 (housekeeping) and GLUD2 (nerve tissue-specific) genes, respectively. These forms differ in their catalytic and allosteric properties. Kinetic studies showed that the K(m) value for glutamate for the nerve tissue GDH is within the range of glutamate levels in astrocytes (2.43 mM), whereas for the housekeeping enzyme, this value is significantly higher (7.64 mM; P<0.01). The allosteric activators ADP (0.1-1.0 mM) and L-leucine (1.0-10.0 mM) induce a concentration-dependent enzyme stimulation that is proportionally greater for the nerve tissue-specific GDH (up to 1,600%) than for the housekeeping enzyme (up to 150%).

Work at the protein level showed that glutamate dehydrogenase purified from human brain consists of 4 electrophoretically distinct isoproteins. These are differentially distributed in the two catalytically active isoforms of the enzyme. Concurrent molecular biological studies showed the presence of four different sized mRNAs and multiple gene copies for glutamate dehydrogenase in the human, thus suggesting a genetic basis for the multiplicity of this protein.

To date, however, only one cDNA encoding for human glutamate dehydrogenase is known. This derives from an intron containing gene that maps to human chromosome 10. Glutamate dehydrogenase (GDH) catalyzes the oxidative deamination of glutamate to alpha-ketoglutarate using NAD or NADP as cofactors. In mammalian brain, GDH is located predominantly in astrocytes, where it is probably involved in the metabolism of transmitter glutamate. In mammalian brain, GDH is located predominantly in astrocytes, where it is probably involved in the metabolism of transmitter glutamate. Glutamate dehydrogenase (GDH) is essential to glutamate metabolism Glutamate dehydrogenase is partially deficient in patients with heterogeneous neurological disorders characterized by the degeneration of multiple neuronal systems (Plaitakis, A., Handb. Clin. Neurol. 1991, 16: 551-568).

Dysregulation of glutamate metabolism occurs in such patients with multiple system atrophy and is thought to cause neuroexcitotoxic nerve cell death (Plaitakis A et al., Science 1982, 216: 193-196). The brains of these patients show loss of glutamate receptors (Tsiotos P et al. Brain Res. 1989, 481: 87-96; Albin R L et al., Brain Res. 1990, 522: 3745) and a selective atrophy of regions that receive glutamatergic innervation (Huang Y P et a/., Ada Neurol. 1984, 41: 39-81), The brain and CNS as such are normally rich in glutamate dehydrogenase immunoreactivity (Aoki C et a/., J Neurosci. 1987, 7: 2214-2231).

When used together at lower concentrations, ADP (10-50 mM) and L-leucine (75-200 microM) act synergistically in stimulating GDH activity. GTP exerts a powerful inhibitory effect (IC(50)=0.20 mM) on the housekeeping GDH; in contrast, the nerve tissue isoenzyme is resistant to GTP inhibition. Thus, although the housekeeping GDH is regulated primarily by GTP, the nerve tissue GDH activity depends largely on available ADP or L-leucine levels. Conditions associated with enhanced hydrolysis of ATP to ADP (e.g., intense glutamatergic transmission) are likely to activate nerve tissue-specific GDH leading to an increased glutamate flux through this pathway.

### OBJECT OF THE INVENTION

It is an object of embodiments of the invention to provide treatment regimes for neurodegenerative diseases such as ALS.

### SUMMARY OF THE INVENTION

It has been found by the present inventor that certain progenitor mesenchymal cells or chondroblast precursor cells show evidence of increased Nrf2 activity and also appear to show reduced or no activity of Keap 1. The same cells also exhibit evidence of increased activity of certain downstream enzymes activated by Nrf2-ARE, that is, the cells show evidence of increased activity of Heme Oxygenase HO-1, thioredoxin and Thioredoxin reductase, glutathione S transferase, Vimentin, and/or tyrosine kinase. Further the cells show no significant presence of neuron-specific glutamate transporter enzymes.

Several mammal stem cells including human stem cells, especially of mesenchymal origin, showing no evidence of impairment of Nrf2 activity or unforeseen increase of Keap 1, may be obtained (harvested) from sources such as the umbilical cord tissue (especially Wharton's jelly), umbilical cord blood, mesenchymal stem cells or progenitor cells from cartilage, such as for instance articular hyaline cartilage and osteoarthritic articular cartilage, mesenchymal stem cells from fatty tissue, from skin and from muscles, and from astrocytes. For the purpose of selection of particularly useful stem cells, they will also be subjected to immunohistochemical methods that evidences nuclear import of Nrf2, e.g. by using an anti-Nrf2 antibody as part of a suitable assay. It is also anticipated that Nrf2 build into a vector system may be transfected into cells for use in the invention (which would then include harvested astrocytes showing impairment of Nrf2 production). The Nrf2 transfection may even create “Nrf2 overexpressing cells”, creating a high probability of translocation of Nrf2 into the nucleus of said cells, thus overriding any possible Keap 1 activity.

The present invention thus relates to significant expression of certain factors in stem cells and more specifically in cells derived from mesenchymal stem cell pools, of Nrf2, an intracellular transcription factor, which again activates several downstream factors such as Heme Oxydant, (HO-1), thioredoxin, thioredoxin reductase, NADH-ubiq.oxidoreductase,

(NQO-1), which appears to activate release of glutathione and of glutamate dehydrogenase, while other factors such as genes/mRNAs for Keap (Kelch-like) protein, and Proteolipid-1 protein (PLP) are not activated at all in these cells.

The concert of gene/mRNA activation and in some cases no inactivation of certain genes/mRNAs, as described above will according to the present invention to provide an optimum material for replacing brain cells, such as astrocytes and astroglia, because said stem cells will be able to take over the antioxidant protection of these cells via released glutathione and at the same time control any surplus of released glutamate (which is otherwise toxic for neurons and/or other brain or CNS related cells). Together with the above described factors this provides for activation of glutamate dehydrogenase capable of inhibiting the buildup of too high levels of glutamate in the brain and/or CNS.

The invention hence concerns utilization of the unique capabilities of certain stem cells isolated from for instance “adult” stem cells as well as isolated from “younger” stem cells from umbilical blood, from placenta or from amnion fluid. The invention also utilizes the detection of the activity of gene/mRNAs for a series of factors such as Nrf2, which in turn activates several downstream factors such as Heme Oxydant, (HO-1), thioredoxin, thioredoxin reductase, NADH-ubiq.oxidoreductase, (NQO-1), which appear to activate release of glutathione and of glutamate dehydrogenase. In some stem cells there may be an additional increase in VEGF activation, which in at least some brain disorders may be of benefit, when transplanted into the CNS system.

Stem cells exhibiting either or all of the above described features are, according to this invention identified for subsequent transplantation into the Central Nervous System (CNS) in areas selected in order to allow the transplanted cells to act as repair cells for impaired CNS cell systems otherwise incapable of rendering gene/mRNA activity of any of the above mentioned proteins/factors in the area selected in the CNS system. When transplanting stem cells to the CNS system, different routes may be chosen, either by cautious and accurated introduction into certain region(s) of the CNS system. This can for instance be accomplished by “coating” the stem cells onto microcarriers, or carriers in nano scale sizes. The stem cells may for instance be adhered to microcarriers made from MPEG PLGA carriers (Coloplast A/S, Holtedam 3, 2970 Humlebaek, Denmark).

The cells may also be administered intrathecal̂ as found appropriate, due to possible “homing effects” exhibited by the cells or alternatively, other modes of administering the cells is anticipated to be used, both using injection or infusion into the CNS, or even by infusion of stem cells intravenously. In this connection, stem cells may be used in the present invention for treating patients suffering from cerebral palsy and from autism as well as hypoxic brain damage in essentially the same way and according to the same types of safety protocols as e.g. those utilized by other researchers and research centers (including the Pediatric Blood and Marrow Transplant Program, Carolina's Cord Blood Bank, Robertson Translational and Cellular Therapy Program, Duke University Medical Center, and the Pediatric Blood and Marrow Transplantation Program, UMC Utrecht), when treating metabolic diseases in children such as for instance metachromatic leukodystrophia. However, according to the present invention, it may be of a significant advantage to obtain effect of the treatment by selecting the stem cells showing activity of Nrf2 and/or any of the downstream enzymes such as for instance HO-1, and further comparing them to markers present on mesenchymal cells such as the presence of CD29 or other identified markers. Cells could be selected for instance by using cell sorting methods (e.g. FACS), followed by expansion of those cells identified prior to infusion or injection of said cells.

Due to the fact that traumatic lesions of the spinal cord appear to cause the astroglia (and other cells) in these regions to exhibit the same impairment which sometimes is compared to the reactions happening during ALS, it may also be possible to use mesenchymal stem cells for treatment of such injuries. The stem cell may be transplanted locally, e.g. together with or coated to microcarriers or to other types of scaffolds that can be placed at the place of injury, or they may be administered intrathecal̂ or simply injected into the spinal fluid. It cannot be excluded that even intravenous injections of stem cells may allow the injected cells to cross the brain blood barrier and work that way.

It is also within the scope of this invention to utilise autologous stem cells in the treatment of neurodegenerative diseases into the CNS system, or allogeneic stem cells from cord blood, in a manner analogous with the use of other mesenchymal stem cells. Autologously or allogeneically derived stem cells offer the possibility of avoiding adverse rejection reactions, even when a complete match is not obtained.

So, it is within the scope of this invention in one or another way to restore astrocyte and/or astroglial cells, using either stem cell approach, or by supplying impaired astrocytes with genes making them capable of repair the previously impaired mechanism, enabling to restore their protective role towards the nerve cells. So, as an alternative to transplantation of stem cells, the present invention also provides the option of a gene therapeutic approach, where astrocytes/astroglia cells are transfected with the about discussed genes, in particular the Nrf2 gene.

A further alternative according to this invention is to activate impaired astrocytes/astroglia cells with inducers of Nrf2 as a mechanism of treatment.

It is also within the scope of this invention to integrate a nucleic acid fragment encoding Nrf2 into a suitable expression vector, which is be tolerated by mammals including man. Such a vector is useful for the treatment of astrocytes in the brain. Alternatively, the vector may be used to transform astrocytes harvested from the brain and inoculated with said vector so as to re-establish the Nrf2 altered activation or lack of activation in astrocytes from patients suffering from neurodegenerative diseases. Thereby, astrocytes that have been damaged or whose function has changed so that they can no longer exert their protective effect (cf. above) can be “salvaged” by ending up with increased glutathione as for instance detected by the re-appearance of glutathione transferase, and at the same time showing decrease of the glutamate concentration in the brain and CNS system.

Such vectors containing Nrf2 encoding material can be administered as injections in the brain at a location where the astrocytes are no longer exhibiting the above-discussed enzyme cascade for instance due to an alteration in their ability to activate Nrf2. Such vectors can typically build on polypeptide chain elongation factor 1a (EF-1a), e.g., a pEF vector harboring a human EF1a promoter instead of commonly used viral promoters. However, according to the present invention, any vector commonly known in the art to be suitable in gene therapy in humans is useful in the present invention, including those vectors that include non-human promoters and/or enhancers as well as other gene expression control regions.

It is known that it is extremely difficult to experimentally increase glutathione content in the CNS in vivo. According to this invention an increased astrocytic glutathione secretion might be obtained by transplantation/injection of stem cells that with respect to this particular feature are capable of functioning as an intact astrocyte and thereby could modify astrocyte-motor neuron interaction by a mesenchymal cell-motor neuron interaction and in part account for motor neuron preservation. This may, both in animal studies and in human clinical trials prevent the initial or the following da mage caused by mutant hSOD 1 within motor neurons that otherwise might attenuate glial activation (Barbeito L H et a/. Brain Res Brain Res Rev 2004, 47: 263-274; Yamanaka K et al., Nat Neurosci 2008, 11: 251-253). In addition, increased glutathione secretion from the transplanted mesenchymal cells might also alter the way in which they interact with other cells and contribute to a decreased microglia response.

The invention hence includes within its scope the provision of therapies using cells exhibiting expression of Nrf2 encoding material, which is sufficient for the Nrf2 to be translocated to the nucleus, combining with ARE, thus resulting in activation of downstream enzymes that effect release of glutathione as evidenced by increased glutathione S transferase, without Nrf2 being inhibited by excess of INrf2 (called Keap 1). The cells, or human cells and more specifically human stem cells, and especially found among the mesenchymal stem cells which can be used to repair the impaired astrocytes in certain geographic areas of the brain, thus prevent or minimize toxic effects on neurons.

The invention also provides for selection of those stem cells that exhibit Nrf2 activation capability sufficient to enable translocation of Nrf2 to nucleu and combine with ARE, ultimately resulting in release of glutathione, and at the same time most probably showing increase of glutamate dehydrogenase to indirectly lower the toxicity by lowering the concentration of glutamate thus avoiding toxic levels of glutamate.

So, in a first aspect the present invention relates to a method for treatment of neurodegenerative disease in a subject in need thereof, comprising administering to the subject cells that express genetic material encoding a therapeutically effective amount of Nrf2. In preferred embodiments the cells administered are stem cells, typically those of mesenchymal origin. As detailed herein, such stem cells may be obtained from a number of suitable sources, and typically the stem cells are selected from the group consisting of stem cells derived from connective tissue (including cartilage), peripheral blood, bone marrow, umbilical cord blood, from placenta, and from amnion fluid.

In certain embodiments of the present invention, the (stem) cells administered are autologous or allogeneic cells, thus minimizing potential problems related to rejection by the recipient of the cells.

Also, in certain embodiment of the present invention, the cells administered have been previously transformed ex vivo with genetic material encoding Nrf2. This embodiment may ensure marked overexpression by the cells of the Nrf2 or at least ensure that cells (such as astrocytes) regain their ability to produce Nrf2 in amounts that suffice for neuroprotection.

In some embodiments the cells are administered so as to settle in close vicinity to impaired cells in the brain or the CNS, where said impaired cells (such as astrocytes or astroglia) have reduced capacity for protecting neurons from degradation, and whereby said cells can take over the protecting function of said impaired cells. This embodiment thus often relates to direct transplantion/positioning of cells into the CNS by methods conventionally used in the art. It is to be understood, however, that other embodiments rely on administration to other sites—this is particularly of interest when cells are administered that have the ability to migrate (“home”) to the desired target area.

The cells administered according to the invention may exhibit further characteristics:
they may conveniently express genetic material encoding a therapeutically effective amount of any one of Heme Oxygenase HO-1, thioredoxin, Thioredoxin reductase, glutathione S transferase, Vimentin, tyrosine kinase,
conveniently, their level of expression of genetic material encoding Keap 1 protein does not impair the protective function of Nrf2, and/or
they conveniently show no significant presence of neuron-specific glutamate transporter enzymes.

It is to be understood that the cells administered may be administered directly into the CNS, including directly into the brain, or into the spinal fluid or intravenously; the latter two options generally require that the cells are capable of homing to the desired site of action or that they are formulated in a manner, which directs their transport to the site of action.

In another and related 2^{nd} aspect, the invention relates to a method for treatment of neurodegenerative disease in a subject in need thereof, comprising administering to the subject genetic material that encode Nrf2, whereby said genetic material is taken up by the subject's cells in the CNS and expressed to provide for a therapeutically effective amount of Nrf2 in the CNS.

This approach constitutes a gene therapeutic alternative to the first aspect of the invention and generally relies on the ability of a suitable gene therapy vector to establish/re-establish gene expression in cells already present in the CNS. Typically said genetic material is included in a vector which is acceptable for administration to human subjects. Such vectors are well-known in the art. Hence, the genetic material is typically under the control of a inducible or constitutive promoter, preferably a promoter derived from a human and the genetic material is typically also in operable linkage with other genetic expression control elements.

In the method of the 2^{nd} aspect, it is due to synergy convenient to also administered genetic material encoding one or more of Heme Oxygenase HO-1, thioredoxin, Thioredoxin reductase, glutathione S transferase, Vimentin, tyrosine kinase, either as part of a genetic construct that includes the genetic material encoding Nrf2 or as one or more separate genetic constructs.

The neurodegenerative disease treated (or at least ameliorated) by means of the methods of the 2 aspects of the present invention are discussed in detail herein—ALS is the primary example, but also multiple sclerosis and other neurodegenerative diseases discussed in the present application are suitable diseases to target with the presently disclosed therapies. Also, trauma-induced injuries in the CNS (e.g. in the spinal cord) may be treated according to the principles of the present invention.

It will be understood that the invention also relates to a cell discussed above as useful for administration for use in a method of the first aspect of the invention. Also, the invention relates to genetic material discussed as useful in the 2^{nd} aspect for use in a method of the 2^{nd} aspect of the invention.

Finally, the invention also relates to use of cells that express genetic material encoding Nrf2 for the preparation of a pharmaceutical composition for use in a method of the first aspect of the present invention. Also, the invention also relates to use of genetic material that encode Nrf2 for the preparation of a pharmaceutical composition for use in the 2^{nd} aspect of the invention.

### LEGENDS TO THE FIGURE

FIG. 1: Human glutamate dehydrogenase (GDH) mRNA levels in chondroblasts from osteoarthritic (O.A.) and non-osteoarthritic subjects (non-O. A.).

The mRNA levels are significantly elevated (>1 Log), when compared to non-elevated mRNA (borderline) between activation and no activation is arbitrarily set at a read out value<100.

FIG. 2: NRH:quinone oxidoreductase mRNA levels in O.A. and non-O. A. derived mesenchymal cells.

The gene encoding NRH:quinone oxidoreductase 2 (NQ02) is found to be elevated in mesenchymal cells, increasing the borderline for expression as seen in the figure, which would fit well into and mimic the activation of this gene due to stress induced protective reaction of the brain and CNS. It is an example of a battery of defensive genes that leads to detoxification of chemicals and ROS.

FIG. 3: Heme oxygenase HO-1 mRNA levels in O.A. and non-O. A. derived mesenchymal cells. Heme Oxygenase HO-1 is significantly activated, more than 2 Logs, when compared to the borderline between activity and no activity in mesenchymal stem cells such as these precursor cells or also called chondroblasts.

FIG. 4: The mRNA encoding NADH ubiquinone oxidoreductase 2 (NQ02) is found to be elevated in mesenchymal cells, increasing the borderline for expression as seen in the figure, which would fit well into and mimic the activation of this mRNA due to stress induced protective reaction of the brain and CNS. It is an example of a battery of defensive genes that leads to detoxification of chemicals and ROS. The presence of this NADH ubiquinone oxidoreductase may therefore also restore the mitochondrial activity in the motor neurons and other brain cells, either preventing further damage to these cells or even enable the transplanted stem cells to repair the mitochondrial activity of cells that still can be induced to be repaired.

### DETAILED DISCLOSURE OF THE INVENTION

### Specific Embodiments of the Invention

### Vector-Promotor Nrf2 Technology to be Used as an Injectible.

Polypeptide chain elongation factor 1a (EF-1a) is a eukaryotic counterpart of E. coli EF-Tu which promotes the GTP-dependent binding of an aminoacyl-tRNA to ribosomes. EF-1cx is one of the most abundant proteins in eukaryotic cells, and expressed in almost all kinds of mammalian cells. A human chromosomal gene coding for EF-1a has been isolated and it has been shown that the promoter of the EF-1a chromosomal gene very efficiently stimulates in vitro transcription (Uetsuki T et al., J. Biol. Chem. 1989, 264: 5791 -5798). A powerful mammalian expression vector, pEF-BOS, has been constructed using the promoter of the human EF-1a chromosomal gene, which very efficiently can stimulate a transcription of proteins such as Nrf2 or other human proteins or enzymes that may be used by in vivo injections into mammals, including humans. The vector system may be obtained from Osaka Bioscience Institute, 6-2-4 Furuedai, Suita, Osaka 565, Japan.

Another group headed by Goldman et al, have modified the vectors, pcDNA1 and pcDNA3, by substituting an EF-1a promoter for the CMV promoter. The resulting vectors, pcDEF1 and pcDEF3, have the high expression capability of the EF-1a promoter, are small (4913 and 6096 bp, respectively) and contain many single cutting restriction sites useful for subcloning genes to be transcribed in human cells and by in vivo injection into mammals, including human vector—promotor carried and activated Nrf2 to be transcribed in vivo in the treatment by effecting Nrf2 transcription in brain cells, in particular in astrocytes or astroglial tissue cells.

Other vector-pro motor systems useful for expressing proteins such as Nrf2 are an integrate part of this invention building on making a new form of injectible concept enabling the intracellular treatment with cytosolic proteins that secondarily may induce several factors ending up improving for instance the healing of chronic ulcers or of beta cell islets in pancreas, as well as cells lacking Nrf2 thus protectin neurons from being impaired or go into apoptosis.

### Stem Cells for use in the Invention

The stem cells showing the above described Nrf2 activation, which in the initial findings is found as mRNA activation. On the other hand Keap (Kelch like protein) appears not to be activated (which means that Nrf2 is not retained in the cytoplasm in an off position caused by Keap). Therefore in the context of this invention, Keap (Kelch like protein) is considered the same as INrf2.

The activated Nrf2 (for instance due to a ROS reaction) enters the nucleus where it binds the Antioxidant Response Element (ARE) that leads to coordinated activation of gene expression. Thus, Nrf2 in the nucleus of the stem cell, together with ARE, is capable of activating some or all of the above-discussed factors as indicated by gene/smRNAs of these particular factors, thereby creating the line of factors enabling the novel concepts and methods for preparing stem cells or precursor cells to provide protection against cellular damage induced chemically or by oxidative stress. Whether the Nrf2 is turned on and off as in the astrocyte is unknown. The Nrf2 ARE acting in the nucleus of the cell actually appears sometimes to be the primary factor that is activated leading to activation of the downstream enzymes etc. ending up in the release of glutathione and release of glutamate dehydrogenase, which again down-regulates the free glutamate down to alpha-ketoglutarate using NAD or NADP as cofactors. This apparently takes place as a response to oxidative processes including ROS, exhibiting a powerful antioxidant effect that again protects the neurons, the motor neurons and other part(s) of the brain and the CNS system and also exhibits antioxidative protection of axons.

All downstream proteins such as Heme Oxydant (HO-1) (NCBI No. Z82244) are activated by activated genes/mRNAs for Nrf2ARE complex in the nucleus, which in turn activates thioredoxin (NCBI No. U78678), and thioredoxin reductase (NCBI No. X91247). At the same time, it is within the scope of this invention that Keap (Kelch like) protein is substantially not expressed in these stem cells, indicating that there is no interaction from that protein, which could decrease the activity in these stem cells, proven by the increased Nrf2 activity in these cells.

Besides expressing the above factors, it is within the scope of this invention to express significant increased levels of glutamate dehydrogenase mRNA (NCBI M20867), which otherwise is decreased in the brain and CNS in patients with neurodegenerative diseases such as for instance ALS so that glutamate in these patients appears in levels toxic to neurons.

As described above, the mesenchymal stem cells, such as for instance chondroblasts, appear in significantly increased levels in patients from which cartilage explants have been obtained. Chondroblasts were obtained by expanding these from small cubes of the cartilage explants cultured as adherent cells in NUNC cell culture flasks, and grown until 70% cell density, and total RNA was harvested for testing on an Affymetrix Gene Chip Analyser at Department for Inflammation Research at Rigshospital (professor Klaus Bendtsen), Copenhagen Denmark.

It appears that mRNA encoded glutamate dehydrogenase is significantly elevated in these cells as can be seen in FIG. 1. It has recently been reported that gradual decrease in GLDH activity may be one of the key factors for neurodegenerative ageing processes (Kravos M, Malesic I. Neurodegener Dis. 2010; 7 (4):239-42). Whereas glutamate dehydrogenase in most mammals (hGDH1 in the human) is encoded by a single functional GLUD 1 gene expressed widely, humans and other primates have acquired through retroposition an X-linked GLUD2 gene that encodes a highly homologous isoenzyme (hGDH2) expressed in testis and brain. Using an antibody specific for hGDH2, we showed that hGDH2 is expressed in testicular Sertoli cells and in cerebral cortical astrocytes (Plaitakis A, Latsoudis H, Spanaki C. Neurochem Int. 2011 September; 59 (4):495-509).

These particular stem cells found among the mesenchymal stem cells, whether they are derived from “adult” stem cells or from “adult” stem cells actually harvested from patients with neurodegenerative diseases such as for instance ALS, harvested from various tissues for instance harvested from fatty tissue, muscle tissue, cartilage tissue, bone marrow, and other tissue outside the brain and the CNS system in such patients suffering from neurodegenerative diseases. It is surprising that even “adult” stem cells from patients with neurodegenerative diseases include stem cells or at least mesenchymal progenitor cells such as chondroblasts, capable of expressing the above described proteins, enzymes, etc.

Alternatively, mesenchymal cells from umbilical cord blood, from placenta, and from amnion fluid, can produce essentially the same factors as the adult stem cells identified among the group of mesenchymal stem cells. It is important that glutamate dehydrogenase mRNA is increased significantly, when such stem cells are intended to be injected or transplanted into patients with neurodegenerative diseases as well as in acute disorders such as spinal injuries. The injected cells can normalize systems in the brain and in the CNS as a whole, by replacing the functionality of brain cells such as astrocytes and astroglia, the system of which seems to be disturbed or destroyed in the brain in patients having neurodegenerative diseases.

The stem cells are identified for transplantation or injection systemically either via intravenous or other vascular route. Alternatively the cells may be introduced into the spinal fluid, to the ventricles of the brain or directly injected into the brain/CNS in areas selected in order to allow the transplanted cells to act as repair cells for impaired CNS cell systems otherwise incapable of rendering gene/mRNA activity of any of the above mentioned proteins/factors in the area selected in the CNS system. The released Glutamate dehydrogenase (GDH) catalyzes the oxidative deamination of glutamate to alpha-ketoglutarate using NAD or NADP as cofactors.

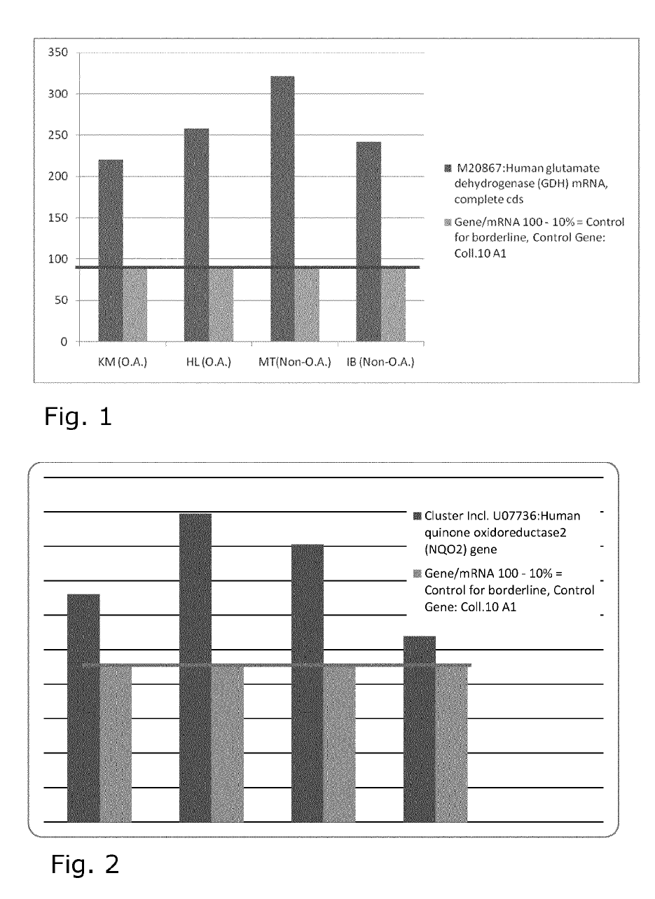

Initial effect of oxidative/electrophilic stress leads to activation of a battery of defensive gene expression that leads to detoxification of chemicals and ROS and prevention of free radical generation and cell survival (FIG. 1). Of these genes, some are enzymes such as NAD(P)H:quinine oxidoreductase 1 (NQO1), NRH:quinone oxidoreductase 2 (NQ02), glutathione S-transferase Ya subunit (GST Ya Subunit), heme oxygenase 1 (HO-1), and γ-glutamylcysteine synthetase (γ-GCS), also known as glutamate cysteine ligase (GCL). Other genes have end products that regulate a wide variety of cellular activities including signal transduction, proliferation, and immunologic defense reactions. The gene encoding NRH:quinone oxidoreductase 2 (NQ02) is found to be elevated in mesenchymal cells (see FIG. 2).

Stem Cell Animal Models

It is within the scope of the invention to select stem cells, typically from the group of mesenchymal cells but also from hematopoietic stem cells (HBSC) that also could contain subgroups of cells that show similar or somewhat similar abilities as mesenchymal cells. The stem cells are selected to express any of the above described mRNAs or genes for factors described herein.

The selected cells from the criteria given above, will be injected either systemically for instance intravenously or by other routes, including intra-spinally, or even directly into the spinal cord with special needle systems, that can be led directly or next to the area where the damaged motor neurons are found to be suffering or to be apoptotic; it would also according to this invention be possible to inject the above described stem cells into certain areas of the brain. These transplantation may in some embodiments be carried out with these cells adhering to a systemic carrier, either as microcarriers or nano carriers, to keep the transplanted cells in place.

These transplanted stem cells can then serve as a source of trophic factors for instance by replacing the impaired astroglia, or astrocytes or other brain derived cells that can restore the impairment and again providing neuroprotection, slowing down neuronal degeneration and disease progression.

SOD1 mice or SOD rats are experimental animals in which the known development of ALS or ALS like symptoms falls in or around 100 days into the life of these animals. It is anticipated that a number of 10 to 20 SOD animals will be treated according to the present invention and compared with the same strain of SOD1 animals developing symptoms in the same period of time in their life. The measurement of beneficial effect of a transplantation of the cells used according to the present invention can be monitored if the treated SOD1 animals live significantly longer than those in the untreated (control) group of SOD1 animals.

One can also investigate the effect by co-culturing the above-described cells with brain and spinal cord extracts from SOD1(G93A) transgenic rats and mRNA expression of the said factors disclosed in this invention as an integrate part of this invention.

Other neurodegenerative diseases can be targeted using the activated mRNA pathway in mesenchymal cells: For instance, Parkinson's disease can be targeted by injecting the selected mesenchymal cells in or near the area of the corpus striatum.

According to the present invention stem cells showing elevated or active mRNA encoding Nrf2 with release from Keap having a similar downstream activation of factors described above, could be used for the treatment of Parkinson's disease, to replace astrocytes impaired in the region near the corpus striatum. This effect could be shown by using mice with striatum damage due to malonate, exactly as demonstrated by conferring Nrf2 over-expressing astrocytes as shown in the study done by Johnson J A et al., Ann NY Acad Sci. 2008 December, 1147: 61-69). In this manner a probable protection against malonate-induced neurotoxicity in cells derived from corpus striatum or in mice with Parkinson-like symptoms could be established.

According to this invention it is important that several of the Nrf2 activated downstream factors such as for instance HO-1 play a major role as a protection against neurodegenerative diseases. This includes the neurodegenerative diseases ALS, Parkinson's disease and Alzheimer's disease. HO-1 has recently been described as a possible entrance port for the treatment of these diseases. It is within the scope of the present invention that stem cells that express mRNA/DNA encoding HO-1 may be sufficient to be used to treat such diseases such as Parkinson's disease and Alzheimer's disease, and may so in other neurodegenerative diseases, whether or not Nrf2 is activated. Therefore, it is also within the scope of this invention that only one or a few of the factors described are expressed in stem cells or more specifically in mesenchymal stem cells, and that these will be a sufficient source as implanted stem cells for instance targeting areas in the brain, where these particular diseases show degeneration of the brain cells, and which may be capable of stopping the disease or even improve the neurodegeneration in these forms of CNS diseases. It is also possible that polyunsaturated fatty acids such as omega 3 rich fatty acids, such as DHA or EPA, may add to the improvement or help stabilizing the neurodegenerative diseases such as it recently has been evidenced, when viewing the effect of omega 3 fatty acids and their possible penetration into the CNS system in mice (Bousquet M et al., Lipd Res. 2010 Nov. 29).

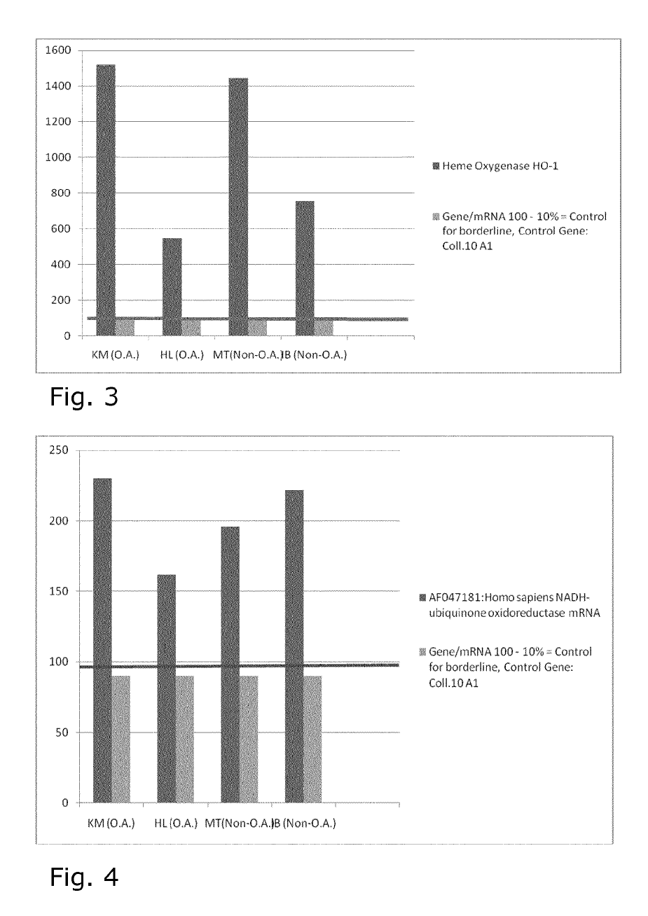

It is possible that some mesenchymal cells show activity for Nrf2 or Nrf2ARE with incorporation into the nucleus of the cells, while other types of mesenchymal cells may express one or more of the other factors described in this invention and may even work in concert, when theoretically all those stem cells or precursor cells expressing one or more of these factors, may be able to elicit a beneficial effect on certain neurodegenerative diseases. One example could be the activity of HO-1 (see FIG. 3), where mRNA/gene encoding HO-1 is extremely active, and may be activated independently without activation of any of the other factors such as for instance the Nrf2 system.

HO-1 participates in the cell defense against oxidative stress and it has been speculated that it might be a new therapeutic target for neuroprotection. In a recent review by Jazwa and Cuadrado (Jazwa A et al., Curr Drug Targets. 2010 December, 11 (12): 1517-31), it is postulated that a drug capable of activating Nrf2, might also be able to indirectly activate HO-1, which then could act as a neuroprotective factor in diseases such as Alzheimers and Parkinsons.

On the other hand The HO-1 gene has been described to be is exquisitely sensitive to oxidative stress and is induced in brain and other tissues in various models of disease and trauma, as shown by Schipper et al (Schipper H M et al., Gupta A, Curr Alzheimer Res. 2009 October, 6 (5):424-30). This group demonstrated that 1) HO-1 protein is significantly over-expressed in AD-affected temporal cortex and hippocampus relative to neurohistologically-normal control preparations, 2) in cultured astrocytes, HO-1 up-regulation by transient transfection of the human HO-1 gene, or stimulation of endogenous HO-1 expression by exposure to beta-amyloid, TNFalpha or IL-1beta, promotes intracellular oxidative stress, opening of the mitochondrial permeability transition pore and accumulation of non-transferrin iron in the mitochondrial compartment, and 3) the glial iron sequestration renders co-cultured neuron-like PC 12 cells prone to oxidative injury. Induction of the astroglial HO-1 gene may constitute a ‘common pathway’ leading to pathological brain iron deposition, intracellular oxidative damage and bioenergetic failure in AD and other human CNS disorders.

Schipper et al.'s hypothesis was that targeted suppression of glial HO-1 hyperactivity according to this group may prove to be a rational and effective neurotherapeutic intervention in AD and related neurodegenerative disorders. To begin testing this hypothesis, studies have been initiated to determine whether systemic administration of a novel, selective and brain-permeable inhibitor of HO-1 activity ameliorates cognitive dysfunction and neuropathology in a transgenic mouse model of AD.

A large body of evidence from postmortem brain tissue and genetic analysis in humans, as well as biochemical and pathological studies in animal models of neurodegeneration suggest that mitochondrial dysfunction is a key pathological mechanism in Parkinson's Disease (PD). The Mitochondrial dysfunction leads to oxidative stress, damage to mitochondrial DNA, mitochondrial DNA deletions, altered mitochondrial morphology, alterations in mitochondrial fission and fusion and ultimately neuronal demise. So, it is also highly probable that the presently presented methods will be able to ameliorate the pathological effects of mitochondrial dysfunction.

It has been reported by Triepels et al that deficiency of NADH: ubiquinone oxidoreductase, the first enzyme complex of the mitochondrial respiratory chain, is one of the most frequent causes of human mitochondrial encephalomyopathies. (Triepels R et a/., Hum Genet. 2000 April, 106 (4): 385-91). NADH: ubiquinone oxidoreductase is the first enzyme complex of the mitochondrial respiratory chain. One can easily understand that if this first enzyme complex is impaired in the brain, neurodegenerative diseases would ensue—already because of impairment of this first enzyme complex in the mitochondria.

Identifying the Mesenchymal Stem Cells with Nrf2 mRNA/Gene Activity Using Alternative Markers

It is known that certain mesenchymal stem cells, among those identified among others with CD73, can be an approach to identify cells which can be used for Islet replacement as a promising approach for type 1 diabetes treatment. As it is well known, the shortage of organ donors demands new sources of β-cells. It is therefore conceivable to isolate these particular stem cells in the tissue of a patient with diabetes type 1, for instance in the fat tissue, in the muscles or may be even in the articular cartilage. These autologous stem/precursor cells may represent an attractive alternative, because they would not be rejected by the patient him/herself. The mesenchymal stem cells (MSC) markers besides CD73 could be CD105, CD90, CD44, CD29, and CD13 that seem to be expressed in mesenchymal cells, as well as nestin and vimentin. With the appropriate stimuli the cells can differentiate into adipocytes and osteoblasts lineages.

It is described that over 13% of em1MCSCs upregulate CD73, a marker of mesenchymal lineage, which can be found in embryonic cell lines. It may be conceivable that a certain percentage of umbilical stem cells or even “adult” stem cells may contain clusters of cells that are CD73 positive and may thus—besides being used for the treatment of diabetes type 1, imitating beta cells, but could also be a possible way of identifying Nrf2 activated cell subsets that can be used to the repair of neurodegenerative diseases.

Other markers such as CD44 may be used to pre-screen the stem cells to be screened for the activity of Nrf2 and thereby, via cell sorting method use such proteins to sort out cells that would benefit as stem cells capable of being used in the treatment of neurodegenerative disease.

A promising approach for increasing antioxidant defences is to transcriptionally increase the activity of the Nrf2/ARE pathway, which activates transcription of anti-inflammatory and antioxidant genes, by simply transplanting stem cells capable of having an already increased Nrf2/ARE pathway as demonstrated in this invention.

Treatment of Neurodegenerative Diseases, by Building at Least Nrf2 into a Vector Harboring a Promoter Tolerable to Mammals including Humans.

As described above the present inventor has found that the Nrf2 mRNA is consistently present and significantly expressed as mRNA identified in four different harvests of articular cartilage from the femoral condyles from four different patients. At the same time it has been described that neurodegenerative disease is associated with decreased function of both motor neurons and sensory neurons that in many cases eventually go into apoptosis or show significant degeneration abolishing the original function of the neuron(s), and depending upon where in the brain or the CNS system the astrocytes have stopped working, these areas should then be inoculated by targeting the impaired astrocytes in order for them to regain their activity.

From this point of view it is within the scope of this invention to create a vector harbouring a promoter directing expression of genetic material encoding Nrf2 in cells such as for instance impaired astrocytes either in vitro or in vivo. This may activate the downstream enzymes such as for instance HO-1, introduced intracellular̂ using technology that utilize human promoters such as for instance EF1a promoter. However, as mentioned above any vector commonly known in the art to be suitable in gene therapy in humans, is useful in the present invention in the system of which Nrf2 is expressed. Such vectors may be capable of being injected to certain specific areas in the brain as for instance substantia nigra in the patient in or near the location of the impaired astrocytes in that particular area.

This may mediate the decreased or impaired Nrf2 and downstream factors and in that manner re-activating the impaired astrocytes, enabling these cells to regain their ability to protect the neurons. The vector-borne genes are applied to for instance astrocytes in various parts of the brain and in the CNS, or to astrocytes harvested from the brain and inoculated with vectors with said proteins followed by re-injection into or near the area from which they were harvested, or in some cases, where the cells can be injected intrathecally.

The intrathecal route of injection according to this invention will also be used both in regards to infuse or transplant stem cells among others, expressing Nrf2, HO-1, etc. or when re-infusing astrocytes previously removed from the patient's brain, inoculated with vector-borne proteins such as for instance Nrf2, HO-1, etc. That intrathecal route may also be used for the injection or infusion of vector borne Nrf2 and/or other factors important, where any or all of the above methods is intended to repair impaired astrocytes in neurodegenerative diseases such as for instances for the treatment of ALS, Parkinson's disease, Alzheimer and/or Huntingdon's disease.

### Experimental

Tests for Elevated mRNA Levels

A DNA chip obtained from Affymetrix with 400,000 fields was used in the evaluation. In the testing done on human chondroblasts, a DNA chip analyzed ˜12,500 different genes in total RNA from 4 chondroblast cultures, of which two different human cell cultures were from osteoarthritic articular (OA) cartilage and two were from biopsies from non-osteoarthritic (non-OA) cartilage (also mesenchymal precursor cells).

Using PCR it was identified that the gene for collagen type X was not expressed in OA. The measurements utilies Affymetrix gene analysis, where in general values below excitation value 100 (an approximate border line value) indicated “no significant gene activity”. Thus any values from gene activity measured which was lower than 1 Log, when compared to a non-expressed gene, was considered non-activated, whereas genes showing >1 Log higher value than 100 was considered activated.

Values obtained from the 4 measurements of collagen type X showed values for OA at 24.2 (patient 1) and 29.2 (patient 2), and for non-OA patients: 54.5 (patient 3) and 76.5 (patient 4). The spreading of values is within or lower than 1 Log difference. The highest of these values were arbitrarily selected from this measurement (76.5) as a control for “no significant” gene activity. This control value was used when comparing excitation values for other genes. When comparing values, for instance for SOX 9 protein (which was found increased using PCR), showing >1 Log compared to the selected control were considered active using the excitation measurement from the chondroblasts from the 4 patients tested. Other examples were genes that are known to be activated in chondroblast cultures: collagen type 1 gene activity, collagen type 2 gene activity. These all were >1 Log higher than the control. This control has been used for estimation of relative gene activity.

Among those, an intracellular transcription factor Nrf2, Nrf2=NF-E2-like basic leucine zipper transcriptional activator, [human, hemin-induced K562 cells, mRNA, 2304 nt, identified from the Affymetrix Gene Chip analysis as NCBI 574017] is elevated in chondroblasts from humans˜mesenchymal precursor cells >1 Log higher than the control (collagen type X), cf. FIGS. 1-4.

On the other hand the Keap 1 transcript was not activated in the four patient samples. The OA patients showed a read out of 15.2 (patient 1), and read out of 21.3 (patient 2), and the non OA patients showed a read out of 12.5 (patient 3), and 15.5 (patient 4). Held together with the borderline value for the control the Keap 1 values are considered as representing “no Keap 1 activity” in this system.

### Tests in a Rat Model

A total of 50 rats at an age of 6 weeks of the SOD1 model of ALS will be purchased from Taconic. The animals will be divided into two groups, one for intrathecal administration of stem cells and one for intrathecal administration of vehicle only. Since only about 60% of the animals will develop both abnormal gait and paralysis it is expected that about 15 animals per group will be included in the statistical analysis. The animals will be weighed at arrival and thereafter once weekly.

At delivery and thereafter daily the animals will be subjected to clinical examinations with regards to abnormalities in their gait. At the first sign of abnormal gait the animals will be treated intrathecally with vehicle or stem cells at the spinal level LI, after laminectomy.

The clinical examinations of gait will continue until the animals reach an age of 250 days or terminated earlier if paralysis is observed. After end of study the animals will be euthanized and the spinal cords recovered for histopathological examination.

After obtaining the results of the histopathological examination the collected data will be included in a draft report which is sent to the sponsor for comments before the final report is issued.

1. A method for treatment of neurodegenerative disease in a subject in need thereof, comprising administering to the subject cells that express genetic material encoding a therapeutically effective amount of Nrf2.

2. The method according to claim 1, wherein the cells administered are stem cells, preferably of mesenchymal origin.

3. The method according to claim 2, wherein the stem cells are selected from the group consisting of stem cells derived from connective tissue, peripheral blood, bone marrow, umbilical cord blood, from placenta, and from amnion fluid.

4. The method according to claim 1, wherein the cells administered are autologous or allogeneic.

5. The method according to claim 1, wherein the cells administered have been transformed ex vivo with genetic material encoding Nrf2.

6. The method according to claim 1, wherein the cells are administered so as to settle in close vicinity to impaired cells in the brain or the CNS, where said impaired cells have reduced capacity for protecting neurons from degradation, and whereby said cells that are administered can take over the protecting function of said impaired cells.

7. The method according to claim 6, wherein said impaired cells are astrocytes or astroglia.

8. The method according to claim 1, wherein said cells further have one or more of the following characteristics:
they express genetic material encoding a therapeutically effective amount of any one of Heme Oxygenase HO-1, thioredoxin, Thioredoxin reductase, glutathione S transferase, Vimentin, tyrosine kinase,
their level of expression of genetic material encoding Keap 1 protein does not impair the protective function of Nrf2,
they show no significant presence of neuron-specific glutamate transporter enzymes.

9. The method according to claim 1, where said cells are administered directly into the CNS, including directly into the brain, or into the spinal fluid or intravenously.

10. A method for treatment of neurodegenerative disease in a subject in need thereof, comprising administering to the subject genetic material that encode Nrf2, whereby said genetic material is taken up by the subject's cells in the CNS and expressed to provide for a therapeutically effective amount of Nrf2 in the CNS.

11. The method according to claim 10, wherein said genetic material is included in a vector which is acceptable for administration to human subjects.

12. The method according to claim 10, wherein said genetic material is under the control of a promoter, preferably a promoter derived from a human.

13. The method according to claim 10, wherein is also administered genetic material encoding one or more of Heme Oxygenase HO-1, thioredoxin, Thioredoxin reductase, glutathione S transferase, Vimentin, tyrosine kinase, either as part of a genetic construct that includes said genetic material encoding Nrf2 or as one or more separate genetic constructs.

14. The method of claim 10, wherein said neurodegenerative disease is ALS.

15. (canceled)

16. (canceled)
